(19) United States
(12) Patent Application Publication
(10) Pub. No.: US 2013/0004545 A1
(43) Pub. Date: Jan. 3, 2013

(54) SLOW RELEASE PHARMACEUTICAL COMPOSITIONS OF ILOPERIDONE

(75) Inventors: Pravin Meghrajji Bhutada, Pune (IN); Sajeev Chandran, Pune (IN); Ashish Ashokrao Deshmukh, Pune (IN); Shirishkumar Kulkarni, Pune (IN)

(73) Assignee: LUPIN LIMITED, Mumbai, Maharashtra (IN)

(21) Appl. No.: 13/518,102

(22) PCT Filed: Dec. 23, 2010
(86) PCT No.: PCT/IB2010/003346

§ 371 Date: Jun. 21, 2012

(30) Foreign Application Priority Data
Dec. 23, 2009 (IN) 1473/KOL/2009

(51) Int. Cl. A61K 31/454; A61P 25/18; A61K 9/00

(52) U.S. Cl. 424/400; 514/321

(57) ABSTRACT

A slow release pharmaceutical composition comprising iloperidone or its active metabolites and a slow release agent is described. Also disclosed is slow release pharmaceutical composition comprising iloperidone or its active metabolites, wherein the slow release composition is a combination of a controlled release composition and an immediate release composition.

### FIELD OF THE INVENTION

The invention relates to slow-release pharmaceutical composition(s), comprising Iloperidone or its metabolite(s) or a pharmaceutically acceptable salt(s), hydrate(s), solvate(s), polymorph(s), stereoisomer(s), ester(s), prodrug(s) and complex(s) thereof, processes of preparing such compositions and their uses.

### BACKGROUND OF THE INVENTION

Iloperidone is a new-generation atypical antipsychotic agent acting as a serotonin/dopamine (5-HT_{2A}/D₂) antagonist, approved by US FDA to Vanda Pharmaceuticals, Inc., for the treatment of schizophrenia, bipolar disorder and other psychiatric conditions. Chemically, Iloperidone belongs to benzisoxazole class of compounds, and shows a multiple receptor binding profile, sharing this feature with other atypical antipsychotic agent(s). Administered orally, the active ingredient is highly bound to plasma proteins and extensively metabolized. U.S. Pat. RE 39,198 discloses Iloperidone and its pharmaceutical compositions which can be administered by various routes such as orally, parenterally or intravenously.

Iloperidone is marketed as immediate release tablets by Vanda Pharmaceuticals under the brand name of Fanapt® in various strengths e.g. 1 mg, 2 mg, 4 mg, 6 mg, 8 mg, 10 mg and 12 mg for twice daily administration.

Iloperidone undergoes extensive metabolism and is eliminated by hepatic metabolism. Two predominant Iloperidone metabolites are P88-8991 (referred to as P88 in this invention) and P95-12113 (referred to as P95 in this invention). P88 is an active metabolite with similar receptor affinity as that of parent compound with a t_{1/2} of 25.6 h. Plasma level of P88 is reported to be approximately 1.5 times that of Iloperidone. On the other hand P95 has a t_{1/2} of 25.6 h and shows affinity for only 5-HT_{2A} receptors.

Various patent applications disclose the pharmaceutical compositions of Iloperidone. U.S. patent application 2005/0020632 and U.S. 2009/0176739 discloses optical isomers of P88, their compositions and uses. It does not exemplify any slow release pharmaceutical compositions of P88.

U.S. patent application 2009/0306137 discloses method of treating depressive illness using Iloperidone or its metabolite. It is also disclosed that Iloperidone and its metabolite can be administered as various pharmaceutical compositions one of them being controlled or pulsatile release. Dissolution profiles are also disclosed.

U.S. patent application 2010/0273809 discloses method for treating prostate enlargement in a male mammal by administering an effective amount of 4-[3-[4-(6-fluoro-1, 2,-benzisoxazol-3-yl)-1-piperidinyl]propoxy]-3-methox-y-benzoic acid (P95) or a pharmaceutically acceptable salt thereof. Controlled release compositions of P95 are disclosed.

WO 2009/076664 discloses method for treating in an animal suffering a condition mediated by 5-HT_{2A} receptors, by administering an effective amount of 4-[3-[4-(6-fluoro-1, 2,-benzisoxazol-3-yl)-1-piperidinyl]propoxy]-3-methoxy-benzoic acid (P95) or pharmaceutically-acceptable salt thereof. Controlled release compositions of P95 are disclosed.

U.S. patent application 2008/0069878 discloses compositions comprising solid dispersion of weakly basic drugs and a timed pulsatile release (TPR) coating. Further, compositions comprising combination of TPR and immediate release beads are also disclosed. Iloperidone TPR composition has been exemplified with dissolution profile which has a lag time.

Major adverse effects of Iloperidone are dizziness, somnolence and cardiovascular side-effects (orthostatic hypotension—change in blood pressure and heart rate).

Active ingredient(s) that are administered in the form of conventional compositions become available to body fluids at a rate that is initially very high, followed by a rapid decline. For many active ingredient(s), this delivery pattern results in a transient overdose, followed by a long period of under dosing. This is a pattern of limited clinical usefulness. The delivery pattern can be improved with the introduction of a variety of slow release systems.

By providing relatively constant, slow release of active ingredient(s) over an extended period of time, the problems of transient overdosing caused by the initial spike in concentration entering the blood stream immediately after administration and the subsequent under dosing can be avoided and controls the dosage to safer and more effective levels.

Thus there is a need for the development of slow release pharmaceutical composition(s) that provides a reliable and reproducible dissolution and plasma concentration profile of the active ingredient, which may reduce the occurrence of various adverse effects of Iloperidone or its active metabolite(s) or a pharmaceutically acceptable salt(s), hydrate(s), solvate(s), polymorph(s) stereoisomer(s), ester(s), prodrug(s) and complex(s) thereof and thus improves patient compliance. These slow release systems thus provide effective medication with reduced side effects, which may be achieved with reduced dosing frequency.

### SUMMARY OF THE INVENTION

The invention relates to slow release pharmaceutical compositions, comprising Iloperidone or its metabolite(s) or pharmaceutically acceptable salt(s), hydrate(s), solvate(s), polymorph(s), stereoisomer(s), ester(s), prodrug(s) and complex(s) thereof, processes of preparing such compositions and their uses.

In another embodiment the slow release pharmaceutical composition(s) comprises a slow release matrix comprising one or more active ingredient(s).

In another embodiment the slow release pharmaceutical composition(s) comprises an immediate release matrix comprising one or more active ingredient and the slow release is achieved by a release modifying coating.

In yet another embodiment an osmotically driven release system can also be used to achieve a slow release.

In another embodiment the slow release pharmaceutical composition(s) provides immediate release and slow release of active ingredient(s), which can be same or different.

Another embodiment provides multilayer or shell type slow release pharmaceutical composition(s), comprising one or more slow release agent(s) and one or more active ingredient(s) such as Iloperidone or its metabolite(s) or a pharmaceutically acceptable salt(s), hydrate(s), solvate(s), polymorph(s), stereoisomer(s), ester(s), prodrug(s) and complex(s) thereof.

In another embodiment the multilayer or shell type slow release pharmaceutical composition(s), comprises active ingredient(s) such as Iloperidone or its metabolite(s) or a pharmaceutically acceptable salt(s), hydrate(s), solvate(s), polymorph(s), stereoisomer(s), ester(s), prodrug(s) and complex(s) thereof, wherein the active ingredient(s) is released slowly in one or more layer(s) of the multilayer composition by one or more slow release agent(s).

In another embodiment the multilayer or shell type slow release pharmaceutical composition(s) comprises core and one or more outer layers comprising active ingredient(s), one or more slow release agent(s) and/or pharmaceutical excipient(s). The active ingredient(s) and one or more slow release agent(s) can either be in the core and/or one or more outer layer(s).

In another embodiment, the multilayer or shell type slow release pharmaceutical composition(s) comprises core and one or more outer layers, wherein the core comprises one or more active ingredient(s), one or more slow release agent(s) and optionally pharmaceutical excipient(s).

In another embodiment, the multilayer or shell type slow release pharmaceutical composition(s) comprises core comprising one or more active ingredient(s), and optionally pharmaceutical excipient(s) and one or more outer layers, wherein the outer layers comprises one or more slow release agent(s).

In another embodiment, the multilayer or shell type slow release pharmaceutical composition(s) comprises core and one or more outer layers, comprising active ingredient(s) and one or more slow release agent(s) and/or pharmaceutical excipient(s), wherein the core comprises entirely of active ingredient(s).

In another embodiment, the multilayer or shell type slow release pharmaceutical composition(s) comprises inert core and one or more outer layers, wherein the one or more outer layer(s) comprises the active ingredient(s), one or more slow release agent(s) and/or pharmaceutical excipient(s).

In another embodiment, the multilayer or shell type slow release pharmaceutical composition(s) comprises core and one or more outer layers, comprising active ingredient(s) and one or more slow release agent(s) or pharmaceutical excipient(s), wherein at least one slow release agent is hydrophobic or hydrophilic.

Another embodiment relates to multilayer or shell type slow release pharmaceutical composition(s), wherein at least one layer comprises slow release agent(s) and the active ingredient(s) and at least one layer comprises bioadhesive agent.

Yet another embodiment relates to slow release pharmaceutical composition(s), which comprises multiparticulate component such as powder, particles, granules, microspheres, spheroids, pellets or minitablets comprising one or more slow release agent(s), one or more excipient(s) and active ingredient(s) which can be different or same having same or different release rate at different times.

Yet another embodiment relates to slow release pharmaceutical composition(s), which comprises multiparticulate component such as powder, particles, granules, microspheres, spheroids, pellets or minitablets comprising one or more slow release agent(s), one or more excipient(s) and active ingredient(s) which can be different or same having same or different release rate at different times, wherein one of the multi-particulate component can be immediate release component for each active ingredient.

Another embodiment provides slow release pharmaceutical composition(s), comprising one or more slow release agent(s) and one or more active ingredient(s) such as Iloperidone or its metabolite(s) or a pharmaceutically acceptable salt(s), hydrate(s), solvate(s), polymorph(s), stereoisomer(s), ester(s), prodrug(s) and complex(s) thereof, wherein the said composition is retained in the upper part of the gastrointestinal tract and release the active ingredient(s) for prolonged period in the stomach and upper small intestine.

In yet another embodiment Iloperidone or its metabolite(s) or a pharmaceutically acceptable salt(s), hydrate(s), solvate(s), polymorph(s), stereoisomer(s), ester(s), prodrug(s) and complex(s) thereof can be administered either alone or in combination with other pharmaceutical active ingredients effective in the treatment of psychotic disorders such as schizophrenia or bipolar disorders.

Another embodiment relates to the manufacture of slow release pharmaceutical composition(s) for the treatment of psychotic disorders. The slow release pharmaceutical composition(s) of the invention can be manufactured by various methods known in the art such as by dry granulation, wet granulation, melt granulation, direct compression, double compression, extrusion spheronization and layering.

### DETAILED DESCRIPTION OF THE INVENTION

The invention relates to slow-release pharmaceutical composition(s), comprising Iloperidone or its metabolite(s) or a pharmaceutically acceptable salt(s), hydrate(s), solvate(s), polymorph(s) stereoisomer(s), ester(s), prodrug(s) and complex(s) thereof.

The tolerability of Iloperidone can be attributed to rate of absorption and therefore, occurrence of tₘₐₓ. It has been reported that incidences of adverse effect under fasting decreases once steady state plasma levels are attained. Therefore, one of the objectives is to slow the rate of absorption and to delay the tₘₐₓ of the active ingredient by virtue of administering the active ingredient once daily using a slow release delivery system to obtain a plateau like pharmacokinetic profile with plasma concentrations corresponding to steady state active ingredient levels. These slow release pharmaceutical compositions may thus reduce the occurrence of various adverse effects of Iloperidone or its metabolite(s) or a pharmaceutically acceptable salt (s), hydrate(s), solvate(s), polymorph(s) stereoisomer(s), ester(s), prodrug(s) and complex(s) thereof. The slow rate of absorption of active ingredient may reduce the peak plasma concentration and accumulation of the active ingredients in the body by balancing a between rate of absorption and rate of elimination with successive administration.

Further, presently the commercially available Iloperidone pharmaceutical composition are administered twice daily, converting twice daily dosing to once daily dosing will improve patient compliance.

The term “active ingredient” denotes Iloperidone as a mixture of various stereoisomer(s) thereof or as one pure stereoisomer thereof as a free base or in the form of a pharmaceutically acceptable salt(s) or any other form such as its metabolite(s), hydrate(s), solvate(s), polymorph(s), ester(s), prodrug(s), complexes and the like mixtures thereof. The slow release pharmaceutical composition(s) generally comprises up to 24 mg Iloperidone as such or equivalent amount of its pharmaceutical acceptable salt(s), hydrate(s), solvate(s), polymorph(s), ester(s), prodrug(s), complexes and the like mixtures thereof. The dose may vary from vary from about 1 to about 50 mg depending upon the compound employed, the host, the mode of administration and the nature and severity of the condition being treated. For the metabolite P88 the indicated daily dosage is in the range from about 1mg to about 500 mg, conveniently administered in divided doses up to four times a day or in slow release form.

The term “pharmaceutically acceptable salt” of the active ingredient refers to a salt(s) of the active ingredient which is physiologically acceptable for pharmaceutical uses, in particular when administered to mammals and/or humans. Pharmaceutically acceptable salt(s) of this type may be formed, for example, with inorganic or organic acids.

The term, “Iloperidone” includes iloperidone base, any salt(s), hydrate(s), solvate(s), polymorph(s), ester(s), complex(s), clatharate(s) and/or stereoisomer(s). Iloperidone can be crystalline or amorphous in nature.

As used herein, “metabolite of Iloperidone” includes the active metabolites of iloperidone or any salt(s), hydrate(s), solvate(s), polymorph(s), ester(s), complex(s), clatharate(s) and/or stereoisomer(s) the metabolite, which have similar receptor affinity like parent iloperidone or its salt(s), hydrate(s), solvate(s), polymorph(s) stereoisomer(s), ester(s), prodrug(s) and complex(s).

In an embodiment the metabolite P88, due to its similar receptor affinity as that of parent active ingredient, can be orally administered as slow release compositions. The tₘₐₓ of P88 is 6 h which corresponds to the earlier report that cardiovascular side-effects occur within 2-4 h of the occurrence of Cₘₐₓ of Iloperidone (i.e., 4-6 h post oral administration of parent active ingredient).

The term “comprising”, which is synonymous with “including”, “containing”, or “characterized by” here is defined as being inclusive or open-ended, and does not exclude additional, unrecited elements or method steps, unless the context clearly requires otherwise.

The term “core” as used herein may be defined as the part of the composition surrounded by at least a part of the coating or layer. Inert core used herein may be defined as the part of the composition surrounded by at least a part of the coating or layer, which does not comprises active ingredient.

In another embodiment the slow release pharmaceutical composition(s) comprises a slow release matrix comprising one or more active ingredient.

In another embodiment the slow release pharmaceutical composition(s) comprises an immediate release matrix comprising one or more active ingredient and the slow release is achieved by a release modifying coating.

In yet another embodiment the slow release pharmaceutical composition(s) is an osmotically driven release system to achieve a slow release. With a release system of this type, preferably an oral system, at least one, preferably all, surface(s) of the release system, preferably those which are in contact or which may come into contact with the release medium, are semi-permeable, preferably provided with a semi-permeable coating, so the surfaces are permeable to the release medium but substantially, and preferably completely, impermeable to the active ingredient, whereby the surface and/or optionally the coating comprises at least one opening for releasing the active ingredient. This is preferably taken to mean a system in tablet form with a delivery opening, an osmotic pharmaceutical composition core, a semi-permeable membrane and a polymeric part which exerts pressure. A useful example of such a system include in particular the OROS® systems such as Push-Pull® system, delayed Push-Pull® system, Multi-Layer Push-Pull® system, the Push-Stick System and in certain cases the L-OROS®. The other types of osmotic drug delivery systems like elementary osmotic pump systems, controlled porosity osmotic pump systems, osmotic bursting osmotic pump systems, monolithic osmotic system, OROS-CT can also be used to achieve a slow release.

Embodiments and examples of the actual production of osmotically driven release systems can be found in U.S. Pat. Nos. 4,765,989; 4,783,337; 4,612,008 and 6,630,162 the complete disclosures of which are incorporated herein by reference.

In another embodiment the pharmaceutical composition(s) provides immediate release and slow release of active ingredient(s) which can be same or different.

The slow release pharmaceutical composition(s) comprises but are not limited to slow release compositions which can be interchangeably used with modified release, delayed release, pulsatile release, timed release, programmed release, extended release, controlled release, osmotic release, sustained release and gastro-retentive compositions. These compositions may be in different dosage form. The non limiting examples of the dosage form of the slow release composition are tablets, pellets, beads, granules, capsules, microcapsules, tablets in capsules and microspheres, matrix compositions, multilayered or shell-type compositions, microencapsulation and powder/pellets/granules for suspension; granules, spheroids, pellets or microcapsules which are filled into sachets or capsules or may be compressed to tablets. Tablets may be single layered tablets, multilayered tablets, mini tablets, bioadhesive or mucoadhesive tablets, caplets, matrix tablets, tablet within a tablet, osmotic tablets, reservoir type tablets, dispersible tablets, erodable tablets and gastroretentive tablets.

Slow release compositions are any compositions other than immediate release composition. These compositions comprise one or more slow release agent(s) and/or pharmaceutically acceptable excipient(s).

Slow release agents are defined as hydrophilic or hydrophobic agents, which can be polymeric or non-polymeric and which are capable of modulating the rate of release of the active ingredient(s), which can be pH dependent or pH independent. The slow release agent(s) may be natural, semi-synthetic and synthetic agents or mixtures thereof.

The non limiting examples of hydrophobic slow release agents are hydrogenated vegetable oil, purified grades of beeswax; fatty acids; long chain fatty alcohols, such as cetyl alcohol, myristyl alcohol, and stearyl alcohol; glycerides such as glyceryl esters of fatty acids like glyceryl monostearate, glyceryl distearate, glyceryl esters of hydrogenated castor oil and the like; oils such as mineral oil and the like, or acetylated glycerides; ethyl cellulose, stearic acid, paraffin, carnauba wax, talc; and the stearate salt(s) such as calcium, magnesium, zinc, other materials known to the person skilled in the art or the combinations thereof.

Natural slow release agents include but are not limited to proteins (e.g., hydrophilic proteins), such as pectin, zein, modified zein, casein, gelatin, gluten, serum albumin, or collagen, chitosan, oligosaccharides and polysaccharides such as cellulose, dextrans, tamarind seed polysaccharide, gellan, carrageenan, xanthan gum, gum Arabic, guar gum, locust bean gum; hyaluronic acid, polyhyaluronic acid, alginic acid, sodium alginate or combination thereof

Synthetic slow release agents are selected from but are not limited to polyamides, polycarbonates, polyalkylenes, polyalkylene glycols such as poly(ethylene glycol), polyalkylene oxides (PEO), polyalkylene terephthalates, polyvinyl alcohols (PVA), polyvinylphenol, polyvinyl ethers, polyvinyl esters, polyvinyl halides, polyvinylpyrrolidone (PVP), polyglycolides, polysiloxanes, polyurethanes, polystyrene, polylactides, poly (butyric acid), poly (valeric acid), poly(lactide-co-glycolide), poly (ethyleneterephthalate), poly (lactide-co-caprolactone), polyanhydrides (e.g., poly (adipic anhydride), polyorthoesters, poly(fumaric acid), poly(maleic acid), polyvinyl acetate, polystyrene; polymers of acrylic and methacrylic esters; carbomer, carbopol®; celluloses and cellulose derivatives such as methyl cellulose (MC), ethyl cellulose (EC), hydroxypropyl cellulose (HPC), hydroxypropylmethyl cellulose (HPMC), hydroxybutylmethyl cellulose, hydroxyl ethyl cellulose (HEC) sodium carboxymethyl cellulose (Sod.CMC), cellulose acetate (CA), cellulose propionate, cellulose acetate butyrate, cellulose acetate phthalate (CAP), carboxymethyl cellulose, cellulose triacetate, cellulose sulfate sodium salt and blends and copolymers thereof or mixtures thereof.

Polymers can be modified by increasing the number of carboxylic groups accessible during biodegradation, or on the polymer surface. The polymers can also be modified by binding amino groups to the polymer. The polymers can be modified using any of a number of different coupling mechanisms available in the art to covalently attach ligand molecules with bioadhesive properties to the surface-exposed molecules of the polymeric microspheres.

Another group of slow release agents comprises but are not limited to polymers having a hydrophobic backbone with at least one hydrophobic group pendant from the backbone. Suitable hydrophobic groups are groups that are generally nonpolar. Examples of such hydrophobic groups include alkyl, alkenyl and alkynyl groups. A further group comprises slow release agents but not necessarily limited to polymers having a hydrophobic backbone with at least one hydrophilic group pendant from the backbone. Suitable hydrophilic groups include groups that are capable of hydrogen bonding or electrostatically bonding to another functional group. Example of such hydrophilic groups include negatively charged groups such as carboxylic acids, sulfonic acids and phosphoric acids, positively charged groups such as (protonated) amines and neutral, polar groups such as amides and imines. Lectins can be covalently attached to polymers to render them target specific.

The slow release pharmaceutical composition(s) of the invention comprises one or more active ingredient(s) and one or more pharmaceutically acceptable excipient(s) include but are not limited to binders, diluents, lubricants, glidants, pH modifiers and surface-active agents.

The amount of excipient employed will depend upon how much active ingredient is to be used. One excipient can perform more than one function.

Non limiting examples of binders include starches such as potato starch, wheat starch, corn starch; microcrystalline cellulose (MCC), celluloses such as hydroxypropyl cellulose (HPC), hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations there of and other materials known to one of ordinary skill in the art and mixtures thereof.

Fillers or diluents include, but are not limited to dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate or mixtures thereof.

Lubricants may be selected from, but are not limited to, those conventionally known in the art such as magnesium, aluminium or calcium or zinc stearate, polyethylene glycol, glyceryl behenate, mineral oil, sodium stearyl fumarate, stearic acid, hydrogenated vegetable oil and talc or mixtures thereof.

Glidants include, but are not limited to, silicon dioxide, magnesium trisilicate, powdered cellulose, starch, talc and tribasic calcium phosphate, calcium silicate, magnesium silicate, colloidal silicon dioxide, silicon hydrogel or mixtures thereof.

The slow release pharmaceutical composition(s) of the invention may contain a surface-active agent such as surfactants and solubilizing agent. The solubilizing agents are defined as the agents that help the active ingredient to solubilize either in composition or in-situ at the site of absorption or action. Solubilizing agents include but are not limited to surfactants, cyclodextrin and its derivatives, lipophilic substances or any combination thereof.

Non-limiting examples of surfactants include water soluble or water dispersible nonionic, semi-polar nonionic, anionic, cationic, amphoteric, or zwitterionic surface active agents or any combination thereof. Examples of anionic surfactants include but are not limited to alkylbenzene sulfonates, sodium lauryl sulfate, di-alkyl sulfosuccinate, lignosulfonates and the like. Nonionic surfactants may include but not limited to ethoxylated linear alcohols, ethoxylated alkyl phenols, fatty acid esters, amine and amide derivatives, alkylpolyglucosides, ethleneoxide/propyleneoxide copolymers, polyalcolols and ethoxylated polyalcohols, thiols (mercaptans) and derivates.

The preferred surface active agent is copolymers composed of a central hydrophobic chain of polyoxypropylene (poly (propylene oxide)) and polyoxyethylene (poly (ethyleneoxide)) that is well known as poloxamer. However, other agents may also be employed such as dioctyl sodiumsulfosuccinate (DSS), triethanolamine, sodium lauryl sulphate (SLS), polyoxyethylene sorbitan and poloxalkol derivatives, quaternary ammonium salts or other pharmaceutically acceptable surface active agents known to the person skilled in the art.

The surface-active agent, solubilizing agent or surfactant may be added in an amount of 0 to 10 parts by weight to 1 part by weight of active ingredient. The amount of the surface-active agent, solubilizing agent or surfactant is preferably 0.1 to 5 parts by weight 1 part by weight of active ingredient and more preferably 0.2 to 2 parts by weight 1 part by weight of active ingredient.

Other solubilizing agents include but not necessarily limited to vitamin E derivatives; monohydric alcohol esters such as trialkyl citrates, lactones and lower alcohol fatty acid esters; nitrogen-containing solvents; phospholipids; glycerol acetates such as acetin, diacetin and triacetin; glycerol fatty acid esters such as mono-, di- and triglycerides and acetylated mono- and diglycerides; propylene glycol esters; ethylene glycol esters and combinations thereof.

The acidic substances can be added to the slow release composition to enhance the solubility of the active ingredients. The acidic substance can be inorganic or organic. No particular limitation is imposed on the inorganic or organic acidic substance used in the invention. The organic substances are preferably organic acids. The examples of organic acids thereof include higher fatty acid such as tosyllic acid, mesyllic acid, benzoic acid, salicylic acid, tartaric acid, citric acid, fumaric acid, maleic acid and stearic acid. Particularly, mesyllic acid, salicylic acid and citric acid are preferable. The organic acids may be used singly or by mixing two or more.

The organic acid may be added in an amount of 0 to 10 parts by weight to 1 part by weight of active ingredient. The amount of the organic acid is preferably 0.1 to 5 parts by weight 1 part by weight of active ingredient and more preferably 0.2 to 2 parts by weight 1 part by weight of active ingredient.

The slow release pharmaceutical composition(s) of the invention can optionally have one or more coatings such as film coating, sugar coating, enteric coating, bioadhesive coating, diffusion coatings, non-permeable coating, semi-permeable coating and other coatings known in the art. These coatings can be functional or non functional. The functional coating may help slow release pharmaceutical composition(s) to release the active ingredient at the required site of action. In one example, the additional coating prevents the composition from contacting the mouth or esophagus. In another example, the additional coating remains intact until reaching the small intestine (e.g., an enteric coating). Premature exposure of a bioadhesive layer or dissolution of a pharmaceutical composition in the mouth can be prevented with a layer or coating of hydrophilic polymers such as HPMC or gelatin. Optionally, Eudragit of various grades or other suitable polymer may be incorporated in coating composition to retard the release of the active ingredient to ensure active ingredient release in the colon. In an embodiment the coating further comprises the active ingredient.

These coating layers comprises one or more excipients selected from the group comprising coating agents, opacifiers, taste-masking agents, fillers, polishing agents, colouring agents, antitacking agents, pore forming agents and the like.

Coating agents include, but are not limited to, polysaccharides such as maltodextrin, alkyl celluloses such as methyl or ethyl cellulose, hydroxyalkylcelluloses (e.g. hydroxypropylcellulose or hydroxypropylmethylcelluloses); polyvinylpyrrolidone, acacia, com, sucrose, gelatin, shellac, cellulose acetate pthalate, lipids, synthetic resins, acrylic polymers, opadry, polyvinyl alcohol (PVA), copolymers of vinylpyrrolidone and vinyl acetate (e.g. marketed under the brand name of Plasdone®) and polymers based on methacrylic acid such as those marketed under the brand name of Eudragit®.

These may be applied from aqueous or non-aqueous systems or combinations of aqueous and non-aqueous systems as appropriate. Excipients can be included along with the film formers to obtain satisfactory films. These excipients can include plasticizers such as dibutyl phthalate, triethyl citrate, dibutyl sibacate, triacetine, polyethylene glycol (PEG) and the like, antitacking agents such as talc, stearic acid, magnesium stearate and colloidal silicon dioxide and the like, surfactants such as polysorbates and sodium lauryl sulphate, fillers such as talc, precipitated calcium carbonate, polishing agents such as beeswax, carnauba wax, synthetic chlorinated wax and opacifying agents such as titanium dioxide and the like. All these excipients can be used at levels well known to the persons skilled in the art.

Non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc.

Also, coatings of polymers that are susceptible to enzymatic cleavage by colonic bacteria are another means of ensuring release to distal ileum and ascending colon. Materials such as calcium pectinate can be applied as coatings to composition and multiparticulates that disintegrate in the lower gastrointestinal tract, due to bacterial action. Calcium pectinate capsules for encapsulation of bioadhesive multiparticulates are also available.

The slow release pharmaceutical composition(s) of the invention can be coated by a wide variety of methods. Suitable methods include compression coating, coating in a fluidized bed or a pan and hot melt (extrusion) coating. Such methods are well known to those skilled in the art.

Multilayer or shell type slow release pharmaceutical composition(s), comprises one or more slow release agent(s) and one or more active ingredient(s) such as Iloperidone or its metabolite(s) or a pharmaceutically acceptable salt(s), hydrate(s), solvate(s), polymorph(s), stereoisomer(s), ester(s), prodrug(s) and complex(s) thereof.

In another embodiment the multilayer or shell type slow release pharmaceutical composition(s), comprises active ingredient(s) such as Iloperidone or its metabolite(s) or a pharmaceutically acceptable salt(s), hydrate(s), solvate(s), polymorph(s), stereoisomer(s), ester(s), prodrug(s) and complex(s) thereof is released slowly in one or more layers of the multilayer composition by one or more slow release agents. Multilayer and shell-type slow release composition(s) can contain one or more coatings which are free from active ingredients. The multi-layer or shell type slow release pharmaceutical composition(s) can be assembled in several different ways.

In another embodiment, the multilayer or shell type slow release pharmaceutical composition(s) comprises core and one or more outer layer(s), comprising active ingredient(s), one or more slow release agent(s) and/or pharmaceutical excipients. Such composition(s) can also be used to commence release of different active ingredients at different times, by inclusion of different active ingredients in separate layers.

In one embodiment, the multilayer or shell type slow release pharmaceutical composition(s) comprises core and one or more outer layer(s), wherein the core comprises one or more active ingredient(s), one or more slow release agent(s) and optionally pharmaceutical excipient(s).

In one embodiment, the multilayer or shell type slow release pharmaceutical composition(s) comprises core comprising one or more active ingredient(s), and optionally pharmaceutical excipient(s) and one or more outer layer(s), wherein the outer layers comprises one or more slow release agent(s).

In another embodiment, the multilayer or shell type slow release pharmaceutical composition(s) comprises core and one or more outer layer(s), comprising active ingredient(s) and one or more slow release agent(s) and/or pharmaceutical excipient(s), wherein the core comprises entirely of active ingredient(s). Such composition(s) can also be used to commence release of different active ingredients at different times, by inclusion of different active ingredients in separate layers.

In another embodiment, the multilayer or shell type slow release pharmaceutical composition(s) comprises inert core and one or more outer layer(s), wherein the one or more outer layer(s) comprises the active ingredient(s), one or more slow release agent(s) and/or pharmaceutical excipient(s). Such composition(s) can also be used to commence release of different active ingredients at different times, by inclusion of different active ingredients in separate layers. The core may comprise carriers or substrates with or without pharmaceutically acceptable excipient(s). The term “carrier or substrates” as used herein includes sugar spheres, microcrystalline cellulose spheres, or pharmaceutical carriers well known in the art.

In another embodiment, the multilayer or shell type slow release pharmaceutical composition(s) comprises core and one or more outer layer(s), comprising active ingredient(s) and one or more slow release agent(s) or pharmaceutical excipient(s), wherein at least one slow release agent is hydrophobic or hydrophilic.

Another embodiment relates to multilayer or shell type slow release pharmaceutical composition(s), wherein at least one layer comprises slow release agents and the active ingredient and at least one layer which comprises bioadhesive agent(s).

Yet another embodiment relates to slow release pharmaceutical composition(s), which comprises multiparticulate component such as powder, particles, granules, microspheres, spheroids, pellets or minitablets wherein at least one or more of the multi-particulate component comprises one or more slow release agent(s), wherein each multi-particulate component includes one or more excipients and active ingredient(s). Such multi-particulate system can be used to commence release of different or same active ingredient(s) at different times and at different rate(s), by inclusion of different active ingredient(s) or inactive ingredients in separate components. In a preferred embodiment of the above, one of the multi-particulate component can be immediate release component for each active ingredient.

In an embodiment the invention relates to the slow release pharmaceutical composition(s) manufactured and designed to be retained in the upper part of the gastrointestinal tract and to release the active ingredient(s) for prolonged period in the stomach and upper small intestine. The gastric retention of pharmaceutical compositions may be achieved by the mechanisms of bioadhesion, flotation, sedimentation, expansion modified shape systems, by the simultaneous administration of pharmacological agent(s) that delay gastric emptying or by the size of the composition.

“Bioadhesion” is defined as the ability of a material to adhere to a biological tissue for an extended period of time. Bioadhesive properties of bioadhesive agent(s) are affected by both the nature of these agents and by the nature of the surrounding media. Bioadhesive and mucoadhesive can be used interchangeably.

Floating systems or hydrodynamically controlled systems are low-density systems that have sufficient buoyancy to float over the gastric contents and remain buoyant in the stomach without affecting the gastric emptying rate for a prolonged period of time. While the system is floating on the gastric contents, the active ingredient is released slowly at the desired rate from the system. After release of active ingredient, the residual system is emptied from the stomach. Many buoyant systems have been developed based on single and multiple unit systems such as granules, powders, capsules, tablets, laminated films and hollow microspheres.

Floating drug delivery systems are classified depending on the use of two formulation variables: effervescent and non-effervescent systems. Non-effervescent floating compositions use a gel forming or swellable agents.

Swellable agents include, but are not limited to, a crosslinked poly acrylic acid, a poly(alkylene oxide, a polyvinyl alcohol, a polyvinyl pyrrolidone; a polyurethane hydrogel, a maleic anhydride polymer, such as a maleic anhydride copolymer, a cellulose polymer, a polysaccharide, starch, and starch based polymers.

Effervescent floating compositions are prepared with the help of swellable agents and various gas generating agents. The gas generating agent is a compound which generates gas when it is in contact with an acidic medium, such as gastric fluid. Said gas generating agent is selected from the group comprising water soluble carbonates, sulfites and bicarbonates, such as sodium carbonate, sodium bicarbonate, sodium metabisulfite, calcium carbonate, and mixtures thereof. Additionally the gas generating composition may consist of a poly-carboxylic acid agent, such as, citric acid, tartaric acid and mixtures thereof.

In one of the embodiment, the gastric retention of pharmaceutical compositions can be achieved by the size of the composition. When the size of the composition is larger than the diameter of lumen of pylori of stomach, the composition retains in the stomach and releases the active ingredients there. A tablet punch size of more than 12 mm can be used to manufacture these compositions. Different types of compositions can be used to retain it in stomach by this mechanism. Pseudogeometric system is used in the invention to retain the composition in stomach by this mechanism. The composition when comes in contact with the stomach fluid, swells up to a size larger than the diameter of the pylori.

Generally, the gastric emptying time in fasted state is in the range from 0-2 hours and in the fed state from 4-6 hours i.e. food, particularly fatty acids, prevents emptying of the stomach. Thus, it is advantageous to administer the gastro retentive pharmaceutical compositions in fed state.

In the context of the present invention the term “fed state” means that patients take the active ingredient at maximum 4 hours, preferably at maximum 3 hours, more preferably at maximum 2 hours, even more preferably at maximum 1 hour, even more preferably at maximum 30 minutes and most preferably just after meal. In an alternative embodiment the patients may take the composition while eating.

In another embodiment Iloperidone or its metabolite(s) or a pharmaceutically acceptable salt(s), hydrate(s), solvate(s), polymorph(s), stereoisomer(s), ester(s), prodrug(s) and complex(s) thereof can be administered either alone or in combination with other pharmaceutical active ingredient(s) effective in the treatment of psychotic disorders such as schizophrenia or bipolar disorders. The invention thus provides a combination comprising a therapeutically effective amount of active ingredient(s) of the invention and a second active ingredient, for simultaneous or sequential administration.

In one of the embodiment, the total weight of the active ingredient in the slow release composition ranges from 1 to 70% of the total weight of the composition, more preferably 1.5 to 50% of the total weight of the composition.

In one of the embodiment, the total weight of the slow releasing agent in the slow release composition ranges from 1 to 90% of the total weight of the composition, preferably 5 to 80%, more preferably 10 to 70% of the total weight of the composition.

In one of the embodiment, the particles size of the active ingredient (D₉₀) is less than 100 μm, preferably less than 80 μm, more preferably less than 60 μm. The solubility of the micronized active ingredient is increased up to 50% more than the solubility of the unmicronized active ingredient.

In another embodiment, a slow pharmaceutical release composition, wherein the particles size of the active ingredient (D₉₀) is less than 100 μm, preferably less than 80 μm, more preferably less than 60 μm.

In one of the embodiment, the slow release pharmaceutical composition has the following in vitro release rate of the active ingredient, measured by the USP Paddle Method at 50 rpm in 0.1 N HCl at 37° C.: 0 to 30% by weight (based on 100% by weight active ingredient) released after 1 hour; 0 to 40% by weight released after 2 hours; 1 to 50% by weight released after 4 hours; 5 to 60% by weight released after 6 hours; more than 35% by weight released after 12 hours; more than 50% by weight released after 18 hours, and more than 70% by weight released after 24 hours. In one of the embodiment, about 0 to 2.8% w/w of active ingredient is released after 1 hour from the slow release pharmaceutical composition.

In one of the embodiment, about 16 to 30% w/w of active ingredient is released after 1 hour from the slow release pharmaceutical composition.

In one of the embodiment, the slow release composition has the following in vitro release rate of the active ingredient, measured by the USP Paddle Method at 50 rpm in 0.1 N HCl at 37° C. for first 2 hrs, followed by a buffer at a pH of 6.8 at 37° C.: 0 to 30% by weight released after 1 hour; 0 to 40% by weight released after 2 hours; 1 to 50% by weight released after 4 hours; 5 to 60% by weight released after 6 hours; more than 35% by weight released after 12 hours; more than 50% by weight released after 18 hours, and more than 70% by weight released after 24 hours.

In one of the embodiment, slow release compositions exhibit a lag phase (i.e. substantially no release of active ingredient) initially or at any time during release. Preferably, lag phase is in between from about 1 to about 6 hrs, more preferably is in between from about 2 to about 5 hours.

For the above-mentioned indications, the appropriate dosage varies depending upon, the compound employed, the host, the mode of administration and the nature and severity of the condition being treated.

Psychotic disorders include one or more of major depression, obsessive-compulsive disorder, panic disorder, social anxiety disorder, social phobia, post-traumatic stress disorder, premenstrual dysphoric disorder, postpartum depression, major depression, dysthymia, treatment-resistant major depression, treatment-resistant bipolar disorder, and generalized anxiety disorder, or a symptom thereof.

The symptoms include at least one of the following: persistent sad, anxious, or empty mood; feelings of hopelessness; pessimism; feelings of guilt, worthlessness, or helplessness; loss of interest or pleasure in hobbies and activities that were once enjoyed, including sex; decreased energy, fatigue, or being slowed down; difficulty concentrating, remembering, or making decisions; insomnia, early-morning awakening, or oversleeping; appetite and/or weight loss or overeating and weight gain; thoughts of death or suicide; suicide attempts; restlessness; irritability; persistent physical symptoms that do not respond to treatment, such as headaches, digestive disorders, and chronic pain; or any combination of the preceding

Another embodiment relates to the manufacture of slow release pharmaceutical composition(s) for the treatment of psychotic disorders comprising Iloperidone or its metabolite(s) or a pharmaceutically acceptable salt(s), hydrate(s), solvate(s), polymorph(s), stereoisomer(s), ester(s), prodrug(s) and complex(s) thereof. These slow release pharmaceutical composition(s) can be manufactured by various methods known in the art such as by dry granulation, wet granulation, melt granulation, direct compression, double compression, extrusion spheronization, layering and the like. The process of wet granulation includes aqueous or non-aqueous granulation. The production process can readily be adapted to the respective requirements and the desired form of administration by procedures well known to persons skilled in the art.

The production of slow release pharmaceutical composition(s) according to the invention is characterized by high repeatability of the release properties with predetermined release rate of the resulting compositions of Iloperidone or its metabolite(s) or a pharmaceutically acceptable salt(s), hydrate(s), solvate(s), polymorph(s), stereoisomer(s), ester(s), prodrug(s) and complex(s) thereof. The release profile of slow release pharmaceutical composition(s) according to the invention has proven to be stable under storage conditions according to Stability Testing Guidelines and also the bioavailability is adequate.

### EXAMPLES

The following examples serve to illustrate the present invention and preferred embodiments, but do not restrict its scope.

### Example 1

| Ingredients | % Content |
|---|---|
| Iloperidone | 2-20% |
| Mannitol | 3-50% |
| Lactose monohydrate | 10-30% |
| Povidone | 1-10% |
| Sodium lauryl sulfate | 0-10% |
| Magnesium stearate | 1-5% |
| Purified water | q.s. |

Coating (For 2-15% wt. gain)

| | |
|---|---|
| Cellulose acetate | 70-90% |
| Polyethylene glycol | 0.5-10% |
| Triacetine | 2-10% |
| Acetone | q.s. |

Iloperidone mannitol and lactose monohydrate were sifted, mixed and granulated with povidone solution. The granules were dried, sifted, lubricated and compressed into tablets. Cellulose acetate was dissolved in acetone, triacetine and PEG. The compressed tablets were coated with the cellulose acetate solution.

### Example 2

| | |
|---|---|
| Ingredients | % Content |

Active ingredient layer

| | |
|---|---|
| Iloperidone | 2-20% |
| HEC | 1-10% |
| Mannitol | 5-25% |
| PEO | 10-60% |
| Sodium lauryl sulfate | 0-10% |
| Magnesium stearate | 1-2% |
| Isopropyl alcohol | q.s. |

Push-pull layer

| | |
|---|---|
| PEO | 10-50% |
| Mannitol | 10-40% |
| Hypromellose | 10-20% |
| HPC | 2-5% |
| Magnesium stearate | 1-2% |

Coating (For 2-10% wt. gain)

| | |
|---|---|
| Cellulose acetate | 10-30% |
| Dibutyl sebacate | 2-10% |
| Acetone | q.s. |

Iloperidone, mannitol and PEO were sifted, mixed and granulated with HEC solution in IPA. The granules were dried and compressed with a layer of lubricated mixture of PEO, mannitol and hypromellose to prepare bilayer tablets. Cellulose acetate was dissolved in acetone and dibutyl sebacate to prepare coating solution. The compressed bilayer tablet was coated with cellulose acetate solution and drilled an orifice with laser drilling technology.

### Example 3

| Ingredients | % Content* |
|---|---|
| Iloperidone | 2-20% |
| Lactose | 0-50% |
| Microcrystalline cellulose | 0-50% |
| Povidone | 1-10% |
| HPMC | 0-30% |
| Ethyl cellulose | 0-30% |
| Magnesium stearate | 1-5% |
| Purified Water | q.s. |
| Non-functional film coating | For 2-5% wt. gain |

*As percentage of the core tablet weight

Iloperidone, lactose and microcrystalline cellulose were sifted, mixed and granulated with povidone solution. The granules were dried, sifted, mixed with HPMC and ethylcellulose. The above mixture were lubricated, compressed into tablets and then coated with a non functional coating.

### Example 4

| Ingredients | % Content* |
|---|---|
| Iloperidone | 2-20% |
| PVP | 1-10% |
| Carbopol | 0-50% |
| Xanthan gum | 0-50% |
| PEO | 0-25% |
| Microcrystalline cellulose | 0-50% |
| Magnesium stearate | 1-5% |
| Purified water | q.s. |
| Non-functional film coating | For 2-5% wt. gain |

*As percentage of the core tablet weight

Iloperidone, microcrystalline cellulose and PVP were sifted and mixed in RMG with a part of xanthan gum and carbopol. The above mixture was granulated by slugging method. The granules were mixed with PEO and remaining carbopol and xanthan gum, lubricated and compressed into tablets. The tablets were coated with a non functional film coating.

### Example 5

| Ingredients | % Content* |
|---|---|
| Iloperidone | 2-20% |
| Lactose | 10-70% |
| PVP | 1-10% |
| Magnesium stearate | 1-5% |
| Isopropyl alcohol (IPA) | q.s. |

Coating (For 2-15% wt. gain)

| | |
|---|---|
| HPMC | 0-30% |
| Ethyl cellulose | 0-30% |
| PVA | 0-20% |
| IPA + Dichloromethane (DCM) mixture | q.s. |

*As percentage of the core tablet weight

Iloperidone and lactose were sifted, mixed and granulated with PVP solution. The granules were dried, sifted, lubricated and compressed into tablets. The compressed tablets were coated with a solution of combination of hypromellose, ethyl cellulose and PVA in IPA and DCM mixture.

### Example 6

| Ingredients | % Content* | % Content* |
|---|---|---|
| Iloperidone | 2-20% | 2-20% |
| Sugar spheres | 40-65% | — |
| Microcrystalline cellulose spheres | — | 40-65% |
| HEC | 0-10% | 0-10% |
| Hydroxy propyl methyl cellulose | 0-15% | 0-15% |
| Eudragit ® | 0-20% | 0-20% |
| Acetone + IPA mixture | q.s. | q.s. |
| Hard gelatin capsule | 1 | 1 |

*As percentage of the final fill weight

Iloperidone was dissolved in DCM and acetone mixture to prepare a clear solution under stirring in presence of HEC and the solution was loaded on sugar spheres/microcrystalline cellulose spheres by fluidized bed processor. The loaded spheres were coated with a solution prepared by dissolving combination of HMPC and Eudragit® in IPA and acetone mixture. The loaded spheres were filled into capsules.

### Example 7

| Ingredients | % Content* |
|---|---|
| Iloperidone | 2-20% |
| Hydrogenated vegetable oil | 5-15% |
| Microcrystalline cellulose | 10-50% |
| Stearic acid | 1-10% |
| Polyethylene glycol | 5-25% |
| Colloidal silicon dioxide | 1-8% |
| Magnesium stearate | 1-5% |
| Non-functional film coating | For 1.5-3% wt gain |

*As percentage of the core tablet weight

Hydrogenated vegetable oil, polyethylene glycol and stearic acid were melted in preheated steam jacketed vessel at 60-70° C. Sifted iloperidone and magnesium stearate were added to the melted mass under stirring. The heating was stopped but stirring continued for 30-45 min until a uniform mass was formed. The molten mass was cooled, milled and mixed with microcrystalline cellulose, colloidal silicon and magnesium stearate; and compressed into tablets. The tablets were coated with non functional film coating.

### Example 8

| Ingredients | % Content |
|---|---|
| Iloperidone | 2-20% |
| PVP | 5-15% |
| Hypromellose | 10-50% |
| Ethylcellulose | 10-50% |
| Lactose | 10-50% |
| Colloidal silicon dioxide | 0.5-2% |
| Magnesium stearate | 0.5-1% |

Coating (For 2-15% wt. gain)

| | |
|---|---|
| Ammonio methacrylate copolymer | 80-90% |
| Dibutyl sebacate | 5-20% |
| Acetone + IPA mixture | q.s. |
| Talc | 1-5% |

*As percentage of the core tablet weight

Iloperidone and lactose were sifted, mixed and granulated with PVP solution. The granules were dried, sifted and mixed with hypromellose and ethylcellulose followed by magnesium stearate and colloidal silicon and compressed into tablets. The tablets were coated with polymeric coating solution prepared by dissolving ammonio methacrylate copolymer and dibutyl sebacate in acetone and IPA mixture. Talc was used for dusting to prevent adhesion during coating.

### Example 9

| Ingredients | % Content |
|---|---|
| Iloperidone | 2-20% |
| PVP | 1-10% |
| HPMC | 0-60% |
| Eudragit | 0-60% |
| Lactose | 10-60% |
| Magnesium stearate | 1-5% |
| Hydro-alcoholic solution | q.s. |

Swellable floating layer

| | |
|---|---|
| Polyethylene oxide (PEO) | 25-75% |
| Hypromellose | 25-75% |
| Mannitol | 0-25% |
| Colloidal silicon dioxide | 0-2% |
| Magnesium stearate | 0-2% |

*As percentage of the core tablet weight

Iloperidone, eudragit and lactose were sifted, mixed and granulated with PVP solution. The granules were dried, sifted and lubricated with magnesium stearate and compressed with a layer of mixture of PEO, hypromellose, mannitol, silicon dioxide and magnesium stearate for prepare bilayer tablets.

### Example 10

| Ingredients | % Content |
|---|---|
| Iloperidone | 2-20% |
| MCC | 5-25% |
| Citric acid | 5-25% |
| Sodium bicarbonate | 10-30% |
| Carbopol | 5-50% |
| PEO | 5-50% |
| Sodium starch glycolate | 5-50% |
| Colloidal silicon dioxide | 0.5-2% |
| Magnesium stearate | 0.5-2% |

*As percentage of the core tablet weight

Iloperidone, MCC, citric acid and sodium bicarbonate were sifted, mixed. The blend was added and mixed with a mixture of carbopol, PEO and sodium starch glycolate; and granulated by slugging method. The granules were lubricated and compressed into tablets.

### Example 11

| | |
|---|---|
| Ingredients | % Content* |

First layer

| | |
|---|---|
| Iloperidone | 2-20% |
| HPMC | 5-35% |
| Poloxamer | 1-10% |
| Mannitol | 0-10% |
| Colloidal silicon dioxide | 0.5-5% |
| Magnesium stearate | 0.5-5% |
| Purified water/organic solvent | q.s. |

Second Layer

| | |
|---|---|
| HPMC | 25-75% |
| PEO | 25-75% |
| Colloidal silicon dioxide | 0-10% |
| Magnesium stearate | 0-2% |
| Non-functional film coating | For 2-5% wt. gain |

*As percentage of the core tablet weight

Iloperidone, HPMC, poloxamer and mannitol were sifted, mixed and granulated using polaxamer solution. The granules were dried, sifted lubricated and compressed with a layer of mixture of HPMC, EPO, silicon dioxide and magnesium stearate to prepare bilayer tablets. The bilayer tablets were coated with a non functional film coating.

### Example 12

| | |
|---|---|
| Ingredients | Mg/Tablets |

CR Matrix Layer

| | |
|---|---|
| Iloperidone | 10.5 |
| Hypromellose E5 | 20.00 |
| Hypromellose K100 LVCR | 100 |
| Hypromellose K100 MCR | 20 |
| Polyoxyethylene oxide (PEOWSR 303) | 10 |
| Lactose Monohydrate | 95 |
| Microcrystalline cellulose | 120.5 |
| Magnesium stearate | 2.0 |
| Colloidal silicon dioxide | 2.0 |
| Purified water/organic solvent | |

Inert Layer

| | |
|---|---|
| Polyoxyethylene oxide | 20 |
| Hydrogenated vegetable oil | 27 |
| Lactose monohydrate | 15 |
| Hypromellose E5 | 50 |
| Hypromellose K100 LVCR | 35 |
| Aerosil | 1.5 |
| Magnesium Stearate | 1.5 |

CR Coating

| | |
|---|---|
| Ethyl cellulose 7 cps | 6.0 |
| Hypromellose | 6.0 |
| Triethyl citrate | 1.2 |
| Organic solvent | qs |

IR active ingredient Loading Coating

| | |
|---|---|
| Iloperidone | 1.5 |
| Opadry | 18 |
| Aqueous/Organic solvent | qs |

Iloperidone, lactose monohydrate and hypromellose K100LVCR were sifted and granulated with using hypromellose E5 solution. The granules were mixed with sifted Hypromellose K4MCR, Polyoxyethylene oxide, Microcrystalline cellulose and aerosil, lubricated with magnesium stearate and compressed with a lubricated mixture of granules of hypromellose, hydrogenated vegetable oil and lactose monohydrate; polyoxyethylene oxide, hypromellose and aerosil to prepare bilayer tablets using suitable punches. The bilayer tablets were coated with a controlling release coating solution of ethyl cellulose, hypromellose and triethyl citrate in organic solvents system. The coated tablets were further coated with solution of iloperidone and opadry.

Dissolution of example 12 was carried out in 900 ml of 0.1N HCl by a USP dissolution apparatus type II (paddle type) at 50 rpm.

| | % of active |
|---|---|
| Time (hr) | ingredient release |
| 0 | 0 |
| 1 | 12 |
| 2 | 15 |
| 4 | 15 |
| 6 | 20 |
| 8 | 32 |
| 10 | 45 |
| 12 | 60 |
| 16 | 80 |
| 20 | 90 |
| 24 | 100 |

### Example 13

| | |
|---|---|
| Ingredients | Qty (mg/tabet) |

CR active ingredient layer

| | |
|---|---|
| Iloperidone | 12.0 |
| Lactose monohydrate | 95.0 |
| HPMC K100 LVCR | 97.7 |
| HPMC E5 | 20.0 |
| Isopropyl alcohol | q.s. * |
| Dichloromethane | q.s. * |
| Microcrystalline cellulose | 123.0 |
| HPMC K4M CR | 20.0 |
| Polyox WSR-303 | 10.0 |
| Magnesium stearate | 1.3 |
| Aerosil 200 | 1.0 |
| Sub-total | 380.0 |

Pseudo geometric layer

| | |
|---|---|
| Lactose monohydrate | 44.3 |
| Povidone K30 | 7.5 |
| HPMC E5 | 52.5 |
| FD&C Blue#1 | 0.7 |
| Water | q.s. * |
| Hydrogenated Vegetable oil (sterotex) | 27.0 |
| HMPC K100 LVCR | 15.0 |
| Aerosil 200 | 1.5 |
| Mg-sterate | 1.5 |
| Sub-total | 150.0 |
| Total weight (in mg) | 530.0 |

* Evaporates during processing

Iloperidone, Lactose Monohydrate, HPMC K100 LVCR were sifted, mixed and granulated with a solution of hypromellose E5 in a mixture of Isopropyl alcohol and dichloromethane. The granules were mixed with sifted microcrystalline cellulose, HPMC K4M CR, Polyox WSR-303 and a part of aerosil 200 to form active ingredient layer. The pseudo geometric layer granule mixture was prepared by granulating a mixture of lactose monohydrate, povidone K30, and HPMC E5 with purified water; and mixed the granules with HPMC K100M CR and remaining aerosil 200. Both the granule mixtures were lubricated with magnesium stearate and compression into bilayered tablets using suitable punches.

Dissolution of example 13 was carried out in 900 ml of 0.1N HCl by a USP dissolution apparatus type II (paddle type) at 50 rpm.

| | % of active |
|---|---|
| Time (h) | ingredient release |
| 0 | 0.0 |
| 1 | 2.6 |
| 2 | 6.3 |
| 4 | 16.9 |
| 6 | 30.0 |
| 8 | 44.2 |
| 10 | 58.8 |
| 12 | 70.7 |
| 14 | 82.7 |
| 16 | 89.2 |
| 18 | 96.6 |
| 20 | 100.4 |
| 24 | 100.4 |

In-Vivo Relative Bioavailability Data of Iloperidone CR Tablets 12 mg Once Daily (Test) vs. Fanapt 6 mg Administered Every 12 h (Reference)

In a monocentric, open, randomized individual dose two-way crossover trial, the formulation of example 13 as a single tabletted dose of 12 mg iloperidone as test and a commercially available iloperidone formulation (Fanapt®) which is administered twice daily as reference, were administered to twelve healthy volunteers, to determine pharmacokinetic data. The test tablet was administered at 0 hours and reference tablets were administered at 0 and 12 hours. Data was determined experimentally by HPLC analysis.

The essential pharmacokinetic data are shown in the following table: The mean value of the AUCₗₐₛₜ and tₘₐₓ of the in-vivo study was as follows:

| | |
|---|---|
| Reference (Ref) | Test |

| AUCₗₐₛₜ | | AUCₗₐₛₜ | |
|---|---|---|---|
| [ng · hr/ml] | *tₘₐₓ [hr] | [ng · hr/ml] | tₘₐₓ [hr] |
| 138.444 | 3.636 | 137.822 | 9.091 |

*tₘₐₓ: The time point at which peak serum concentration in attained after administration of first dose of Fanapt ®.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

In one of the embodiment, the Cₘₐₓ can be same or lower than the immediate release composition.

1. A slow release pharmaceutical composition comprising iloperidone or its active metabolites and a slow release agent.

2. The slow release pharmaceutical composition of claim 1, wherein the slow release agent is hydrogenated vegetable oil, beeswax, fatty acids, long chain fatty alcohols, glycerides, oils, ethyl cellulose, stearic acid and its salts, paraffin, carnauba wax, talc, polyamides, polycarbonates, polyalkylenes, polyalkylene glycols, polyalkylene oxides, polyvinyl alcohols, polyvinylphenol, polyvinyl ethers, polyvinyl esters, polyvinyl halides, polyvinylpyrrolidone, polyglycolides, polysiloxanes, polyurethanes, polystyrene, polylactides, polyvinyl acetate, polystyrene, polymers of acrylic and methacrylic esters, celluloses, cellulose derivatives or mixtures thereof.

3. The slow release pharmaceutical composition of claim 1, wherein the composition comprises a slow release matrix.

4. The slow release pharmaceutical composition of claim 1, wherein the composition comprises a release modifying coating.

5. The slow release pharmaceutical composition of claim 1, wherein particles size (D₉₀) of iloperidone or its active metabolites is less than 100 μm.

6. The slow release pharmaceutical composition of claim 1, wherein the composition is multilayer or shell type pharmaceutical composition.

7. The slow release pharmaceutical composition of claim 1, wherein the composition is gastroretentive.

8. A slow release pharmaceutical composition comprising iloperidone or its active metabolites, wherein the slow release composition is a combination of a controlled release composition and an immediate release composition.

9. The slow release pharmaceutical composition of claim 8, wherein the controlled release composition comprises a slow release matrix.

10. The slow release pharmaceutical composition of claim 8, wherein the controlled release composition comprises a release modifying coating.

11. A method of treatment of psychotic disorders to a patient in-need thereof, by administering a slow release pharmaceutical composition comprising iloperidone or its active metabolites.

12. (canceled)
